(19) United States
(12) Patent Application Publication
(10) Pub. No.: US 2006/0169796 A1
(43) Pub. Date: Aug. 3, 2006

(54) Air freshener with external wick

(76) Inventors: David Timpson, Centennial Park, AZ (US); Norman D. Hammon, Centennial Park, AZ (US)

Correspondence Address: HOLME ROBERTS & OWEN, LLP, 299 SOUTH MAIN, SUITE 1800, SALT LAKE CITY, UT, 84111, US

(21) Appl. No.: 11/046,151

(22) Filed: Jan. 28, 2005

(51) Int. Cl. A61L 9/04

(52) U.S. Cl. 239/53; 239/36

(57) ABSTRACT

A static air freshener has a frame made fiber board impregnated with a scent or from a frame made of scented plastic beads or a frame having a removable source of scent positioned within an article like a “T” shirt. The “T” shirt is made of material selected to regulate and wick the scent from the source of scent to the exterior of the “T” shirt to position the scent in a desired area and in turn freshen the air in the area. A suspension device like a string or hook is provided to mount the air freshener to a support structure.

### BACKGROUND OF THE INVENTION

1. The Field of the Invention

The present invention relates to a wicking-type air freshener, one use being in a vehicle, and more particularly to an air freshener having a support structure with a source of scent and a miniature clothing item like a miniature “T” shirt placed thereover to function as an external wick to regulate the passage of the scent from the source of scent to outside of the miniature clothing item.

2. Related Art

Air fresheners for use in vehicles are known. They are constructed to release a selected smell, odor or scent into the interior of a space that is typically closed or restricted to retain the scent in the space (e.g., the passenger compartment of a vehicle) over a particular time or useful life. A wide variety of air fresheners are available which allow the user to select a particular scent. Some are configured in different shapes like the miniature shape of a pine tree or in the shape of a certain kinds of hats.

Some air fresheners have a scent-containing portion which is inserted into a frame. U.S. Pat. No. 5,823,432 (Hogan) discloses an air freshener in the shape of selected articles of clothing for visual display. U.S. Pat. No. 5,853,672 (Lorman, et al.) shows an air freshener that has a fan for stimulating air circulation. The fan is activated when the driver steps on the brakes and is deactivated upon release or removal of the foot from the brake pedal.

Other air fresheners for use in homes come in different sizes and shapes. U.S. Pat. Des. 461,885 (Jordi) discloses a wall plug air freshener. U.S. Pat. Des. 432,023 (Fox) shows a clock air freshener.

Some air fresheners that are static (no moving parts) have a frame and an insert which is made of an absorbing material which is typically a non-woven material (e.g., a porous fiber board). Upon placement of the air freshener in a desired location, the scent, aroma or smell impregnated into the absorbing material migrates (believed to be molecular diffusion) into the surrounding air so the user in proximity may enjoy the scent, aroma or smell. Inasmuch as there is full surface interaction between the material with the scent and the surrounding atmosphere where the scent is to be experienced, such as the interior of a vehicle, it is generally believed that the scent dissipates, diffuses or is transported into the surrounding volume more rapidly when it is first exposed. The quantity or amount of scent diminishes over time, generally in what could be viewed as an asymptotic curve approaching depletion or complete dissipation. In effect, one has more scent, aroma or smell than desired at the outset with the time the scent, aroma or smell in the atmosphere is in a range (i.e., parts per million concentration in the surrounding atmosphere) desired being relatively short.

### SUMMARY OF THE INVENTION

An air freshener includes an article of clothing proportionally miniaturized to be suspended decoratively within the cab of a vehicle or any other suitable closed or restricted space. A proportionally miniaturized article of clothing is scaled in size to be substantially smaller than its full size or adult size equivalent. That is, the small size is selected so that it can be suspended from, for example, a rearview mirror post within the cab of a motor vehicle without notable interference with the vision of an operator through or out of the windshield of the vehicle.

If the article to be miniaturized is clothing such as a “T” shirt, an adult male size 38-42 would extend from about 18 to 26 inches across the shoulders. A miniaturized “T” shirt would extend from about two to about four inches across the shoulders and thus would be roughly or approximately from about 1/10^{th} to about 1/20^{th} of each dimension of the full size adult article.

The article may be any suitable item that defines an interior. While the present disclosure discusses a “T” shirt which has an interior, a wide variety of other articles including, but not limited to, long sleeve and short sleeve shirts, golf shirts, sweat shirts, jackets, coats, team jerseys and even trousers or shorts can be presented in miniaturized form. Any suitable article of clothing or other article that can be presented or formed from a porous material as discussed so long as the article has the aforementioned interior as well as an aperture through which structure for suspending the air freshener may extend.

The article of clothing is formed from a material which is porous. That is, the material is selected to have a porosity to regulate the passage of air freshener scent there through. The material may be non-woven (paper-like material) that is suitably porous. Thick fiberboard, while theoretically porous, would not be suitable. Rather, tissue-like or thin non-woven materials (comparable to thin coffee filters or facial tissues) are believed to be porous and are thus within contemplation. Preferably, the article is formed from a woven cloth having a weave which limits or restricts the movement of scent (airborne molecules of the scent) from interior through the cloth to the exterior of the material. Virtually any suitable cotton, cotton blend or synthetic cloth (e.g., nylon) can be used with a cotton/poly-resin blend being preferred.

The air freshener includes a frame sized to fit within the interior of the article and is configured to support the article. The frame has a source of scent which is configured to emanate scent into the interior and through the material to the exterior of the article. A suspension structure is associated with the frame and positioned to extend through the opening in the article. The suspension structure is arranged for suspending the frame from a support structure wherever it may be found including in the cab of a vehicle. In other words, the air freshener may be used in any confined space or closed space. Closets, drawers, lockers (in a dressing room at the gym), locker rooms, small rooms, and the like are within contemplation. Of course, it may be used in train cabs, tractor cabs, truck cabs, airplane cockpits, buses, automobiles, and the like. It could even be used in a vehicle that has open doors and windows so long a circulation of the air therein is not such that the scent, aroma or smell would be transported away so rapidly that it could not be detected by the user. These spaces or areas can be regarded as closed spaces or restricted spaces even though the space is not literally closed. Rather, they are sufficiently defined so that a user can enjoy the benefit of the smell, aroma or scent before it is dissipated away from the area of the user by movement of the air.

In preferred configurations, the frame has a shoulder structure from which the article is suspended. In other preferred arrangements the source of scent is the frame itself which is formed from scented plastic beads. In other arrangements, the source of the scent is a removable element having scent bearing material positioned for contact with the atmosphere. The removable element may be formed from scented plastic beads. The frame desirably has a retaining structure to removeably hold the removable element. Thus, the user can replace the removable element when it is spent or remove one that is undesired to obtain another preferred scent. In more preferred configurations having a removable element, the retaining structure is sized to position the removable element centrally within the interior of the article such as the miniaturized article of clothing.

### DESCRIPTION OF THE DRAWINGS

In the drawings which depict what are presently regarded as preferred embodiments:

FIG. 1 is a perspective view of an air freshener of the present invention;

FIG. 2 is a front view of the air freshener of FIG. 1;

FIG. 3 is a side view of the air freshener of FIG. 1;

FIG. 4 is a top view of the air freshener of FIG. 1;

FIG. 5 is a bottom view of the air freshener of FIG. 1;

FIG. 6A shows a hanger as an alternate to the frame of the air freshener of FIG. 1;

FIG. 6B shows a source of scent for use with the frame of FIG. 6A;

FIG. 6C shows portions of the frame and the source of scent of FIGS. 6A and 6B in cross section and arranged for assembly;

FIG. 7 is a front view of an alternate frame for use with the air freshener of the present invention;

FIG. 8 is a cross section of an article suspended from the frame of FIG. 7;

FIG. 9 is an alternate frame for use with the air freshener of FIG. 1;

FIG. 10 is a front view of an alternate form of an air freshener of the invention;

FIG. 11 is a side view of the removable element of FIG. 10;

FIG. 12 is a front view of an alternate frame with a source of scent of the present invention; and

FIG. 13 is a partial cross sectional view of portions of the frame of FIG. 12.

### DESCRIPTION OF THE ILLUSTRATED EMBODIMENTS

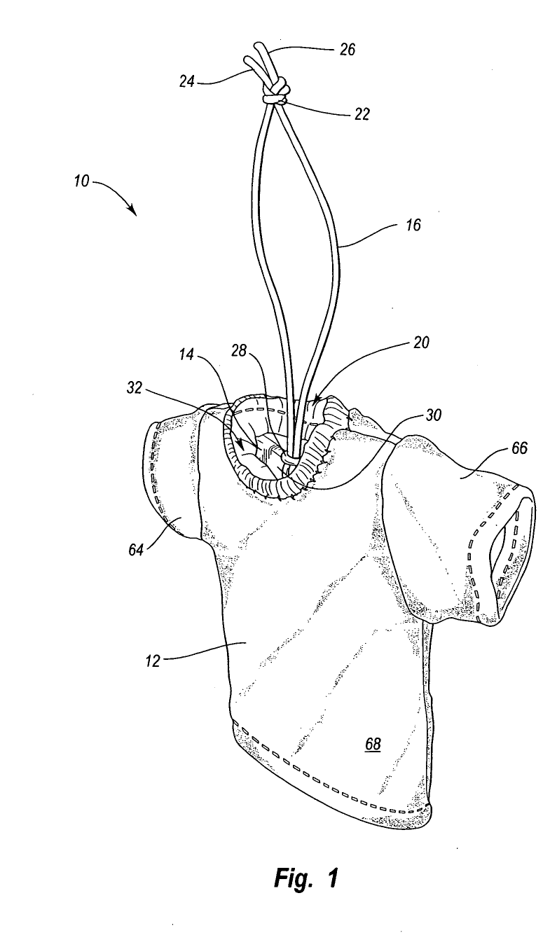

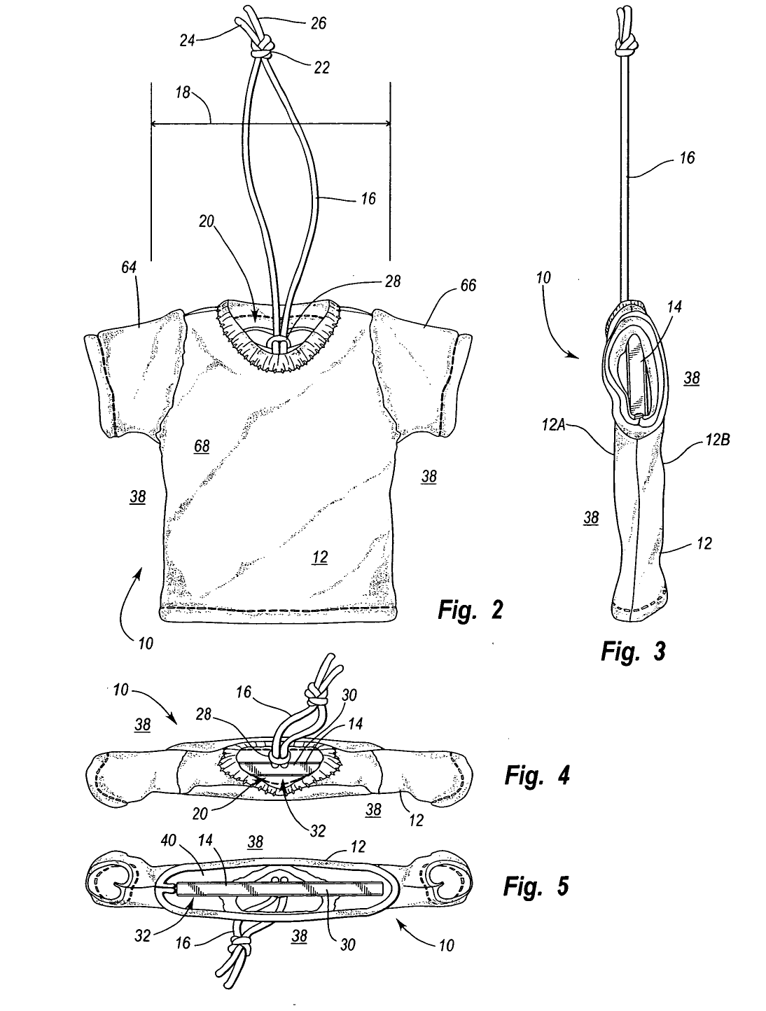

FIGS. 1 through 5 depict an air freshener 10 that includes an article over a frame. More specifically, an article of clothing which is here shown as a miniature “T” shirt 12 is positioned over or supported by a frame 14 to which suspension structure, like string 16, is attached.

The air freshener 10 is principally for use in the cab of a vehicle. In this case the term “cab of a vehicle” is intended to include the passenger compartment of an automobile, a tractor cab, a truck cab, the passenger compartment of a bus and van, the cab of a street car or subway train, the cab of a locomotive, or any other space/volume associated with a vehicle in which users are typically found and in which users may wish to deploy a preferred scent. While the cab of a vehicle may be enclosable by closing doors and windows, other vehicles can have a cab that do not have doors or windows. While the efficacy of the air freshener may be reduced because the scent, odor or smell may dissipate, users may still find sufficient benefit so long as the air movement is such that a user can detect and in turn enjoy the benefit of the scent, aroma or smell from the air freshener.

While the cab of a vehicle is the intended principal use, the air freshener may be used wherever a user desires a scent, aroma or smell, including, but not limited to, and by way of example, a bathroom or kitchen. It could also be used in a closet, a rest room, a locker, a drawer in a chest of drawers, locker room, gym bag, or in any other area or space where a user wants to have a desired scent or odor.

In connection with the cab of a vehicle, typical automobiles and small trucks have some kind of rearview mirror deployed in the middle of the windshield to allow the operator to see through the rearview window. The rearview mirror typically has a post that extends from the windshield or other suitable structure with the rearview mirror attached.

The air freshener 10 is sized to be suspended from the post of the rearview mirror. However, it may of course be suspended or hung from any other suitable structure in the vehicle cab or from any other suitable structure in any other space or area in which the user wants to deploy the air freshener 10. The air freshener 10 is formed or shaped to be a desired decorative article which may be any article that has a front and back with an interior for the frame. The air freshener has a source of scent and the decorative article has an opening through which suspension structure may extend for suspending the air freshener from an external support like the rearview mirror post of a motor vehicle. Of course, the external support may be any kind of hook, button, knob, latch or the like upon which a string or hook can be placed for suspending the air freshener 10.

In this embodiment, the article could be something other than an article of clothing. For example, it could be a miniature tent, suitcase, trash can, bucket or any one of many other objects, so long as it can be made of material that is porous and suspended on a frame. Here, the article is preferably an article of clothing and more specifically a “T” shirt 12. It can be made to be decorative to present desired expressions or images on the front or back and also colored to represent teams and organizations. The article, and more specifically the “T” shirt 12, is proportionally miniaturized to be suspended decoratively within the space in which it is deployed, such as the cab of a vehicle. As noted earlier, it may be suspended from the post of a rearview mirror found in the passenger compartment of an automobile.

Items suspended in the middle of the windshield can interfere with the ability of the operator to see in a line from the operator's seat through the middle of the windshield to outside the vehicle. Thus, use of an article that is small like a miniature “T” shirt is believed to minimize the area that is blocked by the air freshener itself 10, in turn, minimizing the impact on the visibility available to the operator. Of course, a small size for suspension elsewhere in the cab also is desired so that the air freshener 10 does not interfere with other controls or with use of other components within the space/cab.

A miniaturized article of clothing like a “T” shirt 12 is scaled in size to be substantially smaller than its full size or adult size equivalent. A “T” shirt for a male size 38-42 extends from about 18 to 26 inches across the shoulders. A miniaturized “T” shirt is sized to extend from about two to about four inches across the shoulders 18 and thus, would be approximately from about 1/10^{th} to about 1/20^{th} of the shoulder dimension of the full size adult version and similarly about 1/10^{th} to 1/20^{th} of the other dimensions. Similar proportional reductions can be effected for virtually any other item that a user would like to suspend, including long sleeve and short sleeve shirts, golf shirts, sweat shirts, jackets, football, soccer or other team jerseys, coats shorts or trousers.

In FIGS. 1-5, the “T” shirt 12 is shown with an aperture or opening 20 that can be regarded as comparable to the head opening for the head of a user of a regular sized “T” shirt. As can be seen, the suspension structure is any structure or device that allows the user to hook or loop over structure to suspend the air freshener 10. While wire may be used as the suspension structure, a string or line, and more particularly a woven nylon string 16 has been found most suitable as it is decorative while having sufficient strength to resist separation or breaking and having sufficient flexibility to allow a user to form a knot 22 with opposite distal ends 24 and 26. A simple loop hitch 28 allows the string 16 to be attached to the frame 14. While nylon string 16 is preferred for the embodiment illustrated, it should be understood that other types and kinds of string can be used, as well as a wide varied of wires and hooks. Virtually anything that facilitates connection between the frame 14 and a support structure may be used with the string preferred because of its simplicity and flexibility in use.

The frame 14 is here shown to be formed of shoulder member 30 that extends linearly approximately the width 18 of the “T” shirt 12 and upon which the “T” shirt 12 is suspended or hung. The frame 14 can be likened to a miniature clothes hanger with no hook. While the frame 14 typically has rounded edges, the frame 14 may also be rectangular, square, octagonal or triangular, or any other desired geometric shape.

The frame 14 includes scent means, which is here scented plastic beads used in a molding process to form the frame 14. The scented beaded plastic is typically either a polyvinylchoride (pvc) or a polyolefin copoly plastic. Suitable plastic beads for this purpose may be purchased from Dyvex Industries, 30 Enterprise Drive, Carbondale, Pa. The scent is added to the plastic and may be any desired scent. For example, one could select scents that to the user produce aromas or a smell much like pine (tree or cone), new car, lilac, rose, or any other desired smell or aroma. When the plastic beads are molded into the side member, the scent, smell or aroma becomes integral therewith. That is, the scent, aroma or smell is made up of molecules that release from the surface of the frame 14 to the surrounding atmosphere.

Alternately, the frame 14 could be made of a non-woven material such as a scent impregnated paper or paper-like (cellulose) material such as fiber board. Indeed, in some embodiments, scented paper or fiber board is a preferred choice of material for the frame 14 as it is less costly and will release scent, aroma or smell in a fashion similar to plastic formed from scented plastic beads when positioned within the article like “T” shirt 12.

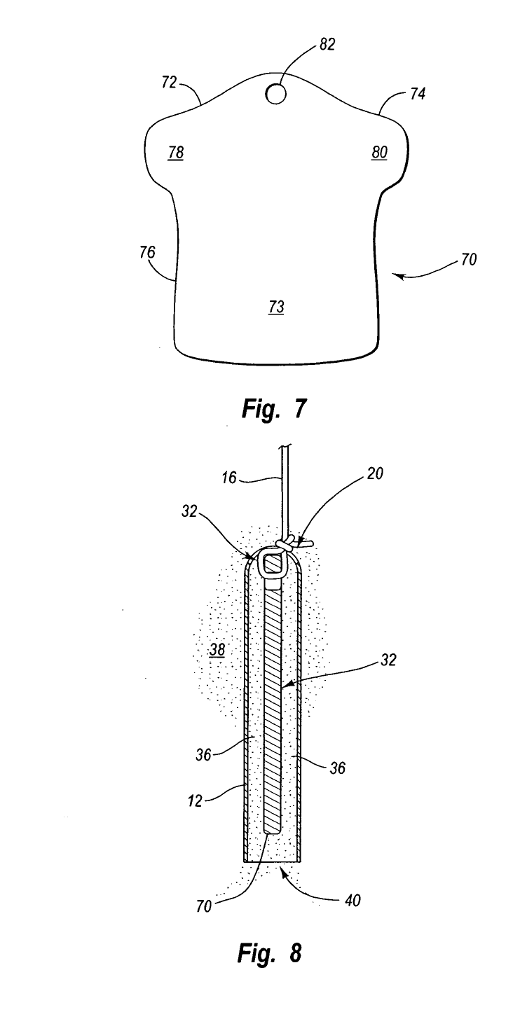

Referring briefly to FIG. 8, the frame 70 of FIG. 7 is shown to be the source of molecules 36 of scent which populate the interior 32 of the “T” shirt 12. The molecules 36 (depicted as dots to illustrate their existence and to represent density) migrate toward the “T” shirt 12 but do not pass through because there is structure (material) that resists the movement of gas/air. However, the material is porous so that some molecules migrate into the “T” shirt 12 and adhere to the material or fibers of the “T” shirt and remain there while others migrate through the “T” shirt 12 to the exterior 38. As the population of molecules in the interior 32 diminishes, it is believed that some of the molecules adhering to the material release and migrate to the exterior 38. Whatever the process, the effect is to extend the time that the scent can be detected by the user for a given amount of scent.

Stated alternately, the population of molecules 36 of scent in the interior 32 is high as the molecules 36 migrate into the interior 32 from the source of scent and more specifically from the frame 70. As the molecules 36 of scent move from the frame 70 into the interior 32, the population of molecules of scent available from the frame 70 to move into the interior 32 diminishes, but at a rate lower than if the “T” shirt 12 is not there. It is believed that the scent molecules 32 seek what could be effectively an equilibrium between the source and the interior 32. Without the “T” shirt (or other article) to impede or restrict the dissipation of scent molecules 36, the scent would rapidly dissipate. However, the “T” shirt 12 slows the dissipation to the exterior 38 and in turn, slows the migration of scent molecules from the frame 70. While some molecules 36 of scent are transmitted through the neck opening 20 and even through the bottom opening 40, the “T” shirt 12 functions to regulate or restrict the dissipation of the scent molecules 36 to outside 38 of the air freshener 10 leading to longer useful life for the air freshener 10 for a fixed amount of scent provided in the frame 70 itself.

With the decreasing population of molecules 36 of scent, aroma or smell, the rate of release to the atmosphere to the exterior 38 diminishes with time. However, by using scented plastic, the amount of scent, aroma or smell that can be included is increased and the amount that is delivered can be controlled so that the air freshener has a longer useful life. That is, the frame 70 can deliver molecules of scent, aroma or smell in sufficient quantity to be detectable by a typical user over a longer period of time in comparison to a comparably sized air freshener that has a scent that is released directly into the surrounding air with no material to regulate the further release of scent.

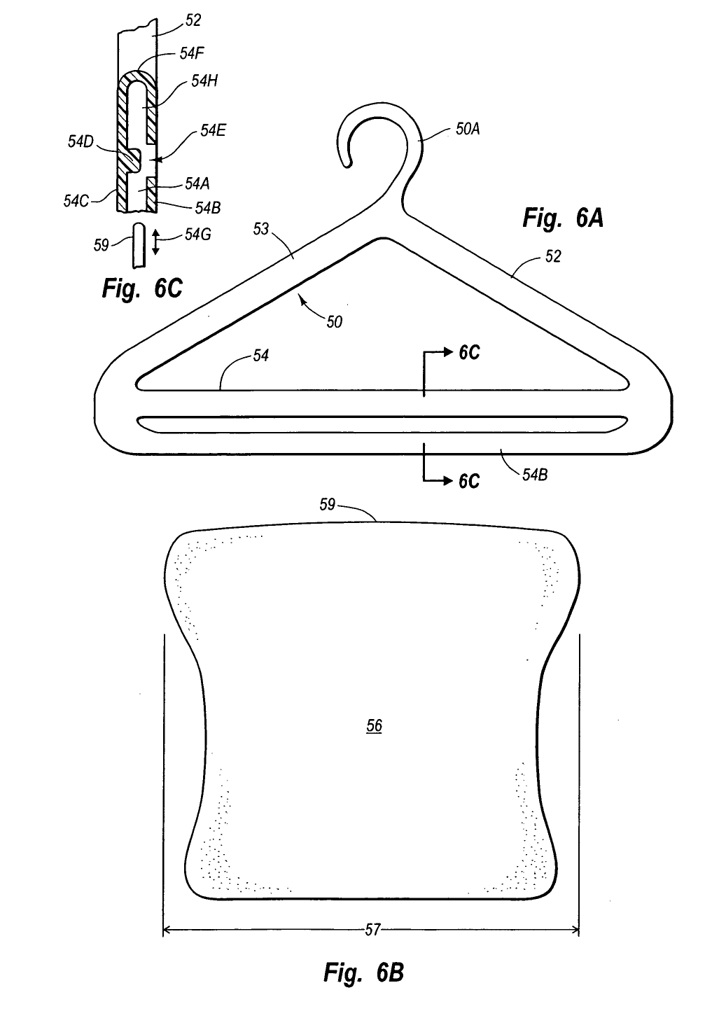

Turning to FIGS. 6A-C, an alternate form of frame 50 is shown which is a miniature coat or pants hanger with a hook 50A. The hook 50A may be in other forms or shapes so long as it works to allow the frame 50 to be suspended from a suitable support. The frame 50 preferably has shoulders 52 and 53 from any suitable plastic without scent and with a pants bar 54 to which a source of scent 56 is attached.

The source of scent 56 to be attached to said pants bar 54 is here shown in FIG. 6B to be rectalinear in shape having a width 57 selected to be less than the width of the pants bar 54. The source of scent 56 is here shown to have an hourglass shape to reflect a female shape useful with certain articles. The source of scent 56 has an upper edge 59. The upper edge 59 is shown in FIG. 6C where it can be inserted into an attaching structure which has a slot or gap 54A between sides 54B and 54C. Side 54C has a nose 54D sized to register with and fit into the slot 54E formed in the side 54B. The sides 54B and 54C can be separated because they are joined at the top 54F and thus, may be elastically deformed or separated to receive the upper edge 59. As the upper edge is inserted 54G into the gap 54A, the nose 54D and side 54C and the side 54B separate. As the edge 59 is urged through the slot 54A, it is urged into the slot 54E and even into cavity 54H. When side 54C is released, it urges the nose 54D toward the slot 54E and in turn the area proximate the edge 59 to thereby retain the edge 59 snuggly and in turn hold the source of scent 56 suspended beneath the frame 50. To remove the source of scent 56, one only need pull down or outward 54G. The nose 54D will deflect to allow passage of the edge 59 out of the slot 54E and out of the gap 54A.

The amount of aroma, smell or scent that is released over a unit of time is controlled not only by the surface area of the source of scent 56 but also by the material from which the article like “T” shirt is formed. That is, the amount of surface area of the source of scent 56 can be varied by simply changing its dimensions and also its shape or form. In turn, the useful life of the air freshener 10 can be regulated and in fact extended for a given amount of scent. That is, the scent emanating from source of scent 56 migrates to and contacts the “T” shirt which adsorbs the scent and wicks it to the exterior surface releasing the scent to the area outside of the “T” shirt or other article.

If the material of the “T” shirt 12 has any elasticity, it can even be sized to be stretched over the frame 70. With the “T” shirt 12 mounted on the frame 70 either loosely or tightly, the air freshener 10 presents a “T” shirt image. That is, the shoulders 72 and 74 with torso section 76 help the “T” shirt 12 retain its “T” shape. The sleeves 78 and 80 make the sleeves 64 and 66 of the “T” shirt 12 extend away from the body or trunk 68 of the “T” shirt 12. In turn, the “T” shirt 12 retains or maintains its “T” shape when on the frame 70. The frame 70 with its torso section 76 also keeps the body or trunk 68 of the “T” shirt 12 extended so that any slogans, sayings or graphics placed on the front 12A and/or back 12B of the “T” shirt (FIG. 3) are more visible. The frame 70 is preferably made of scented beaded plastic or scented fiberboard so that it is itself a source of scent.

The article, and in turn the article of clothing like “T” shirt 12 is made from a material that is selected to have a porosity to regulate the passage of air freshener scent, and more particularly, the scent molecules 36 there through. The material may be a non-woven (paper-like material) that is suitably porous. The density of fibers from which the “T” shirt is made and the arrangement thereof can be selected to allow fluids, and in turn, airborne molecules to pass through. Materials comparable in porosity to thin coffee filters or facial tissues are within contemplation.

Preferably, the article is formed from material that is a woven cloth having a weave which limits or restricts the movement of molecules 36 of scent (dots) from interior through the cloth of the “T” shirt 12 or other article to the exterior 38 of the material as shown in FIG. 8. Virtually any suitable cotton, cotton blend or synthetic cloth (e.g., nylon) can be used with a cotton/poly-resin blend being preferred. Nylon has been used in some embodiments. A preferred composition or blend has about 35% cotton fibers and 65% poly-resin fibers. It is believed that molecules 36 in the interior 32 migrate onto and attach or adsorb onto the material of the article restricting or slowing the dissipation of scent and regulating to the rate the scent molecules migrate or wick to the exterior 38. It is believed that the scent molecules 36 adhere to the nylon fibers to effect the wicking action. At the same time, the material of the article acts as a partial barrier to regulate the movement of scent molecules 36 and, in turn, preferably slow the delivery rate of the scent to the surrounding atmosphere. It is believed further that material from which the article such as “T” shirt 12 is made, regulates the delivery of scent to some extent because a dense or tight weave restricts the passage of molecules 36 from the interior 32 of the article and those less dense or with a looser weave are less restrictive to the passage or wicking of molecules 36 (depicted as dots) from the interior 32 to the exterior 38 as illustrated in FIG. 8.

In FIG. 7, the frame 70 has an upper left shoulder 72, an upper right shoulder 74, and a torso section 76. Shaped as shown, the “T” shirt 12 is stretched thereover to present a “T” shirt image, as hereinbefore discussed. The frame 70 is preferably made of scented plastic beads. The frame 70 also has an aperture 82 to receive a line or string-like string 16 (FIG. 1).

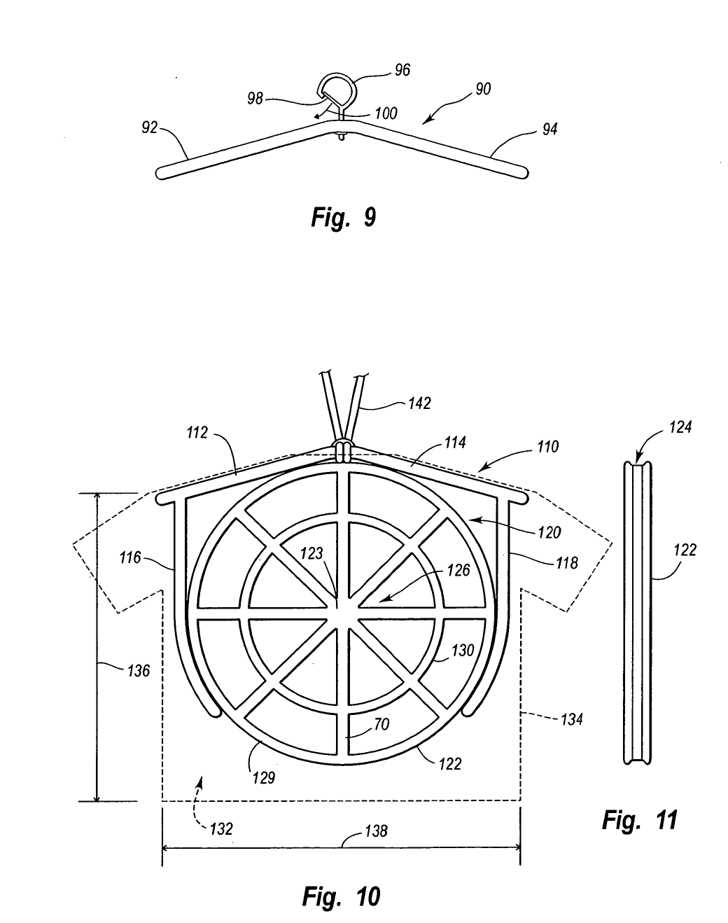

In FIG. 9, a frame 90 is shown that is a kind of miniaturized clothes hanger molded from scented plastic beads. It has opposite shoulders 92 and 94 with a hook 96 centrally attached in a conventional manner. The hook 96 has an optional spring loaded snubbing member 98. That is, it is spring loaded to urge it outwardly 100 so that when the air freshener 10 with such a frame 90 is attached to a support structure, the snubber 98 is urged against the hook 96 to inhibit movement of the hook 96 from a support structure like a rearview mirror post. One must positively urge the snubber 98 inward to make a passage for it to be removed from the support structure. The snubber 98 thus acts to hold the air freshener 10 in place.

FIG. 10 shows a frame 110 with a left shoulder 112 and a right shoulder 114. A left retaining arm 116 and a right retaining arm 118 extend from the left shoulder 112 and right shoulder 114 and are preferably unitarily formed therewith. The left retaining arm 116, the right retaining arm 118, the left shoulder 112 and the right shoulder 114 together define a scent area 120 into which a scented disc 122 is placed. The scented disc 122 is made from scented plastic beads and functions as a source of scent.

The scented disc 122 is also formed to have a groove 124 as seen in FIG. 11. The groove 124 is sized to receive the left side arm 116, right side arm 118, left shoulder 112 and right shoulder 114 fit to hold the scented disc 122. The right side arm 118 and the left side arm 116 are both formed from plastic sufficiently flexible so they can be bent or deflected to allow the user to insert the scented disc 122. Thus, with the scented disc 122 in place, the right side arm 118 and the left side arm 116 urge the scented disc 122 up against the left shoulder 112 and the right shoulder 114 to hold the scented disc 122 firmly in place.

The scented disc 122 is here shown to be circular in projection with an internal lattice work 126 to provide strength. In FIG. 10, the lattice work 126 is shown to have legs 128 every 45 degrees with an outer support ring or rim 129 and one additional support ring 130. Legs 128 can be mounted in whatever spacing desired (15 degrees, 30 degrees, etc.) with additional rings added as well. In addition to strength, the surface area of the scented disc 122 can be controlled by adding or subtracting legs 128 and adding or subtracting rings like ring 130; and in turn, one can control the rate of release of scent molecules to the interior 132 of the “T” shirt 134 (shown in phantom mounted on the frame 110).

In FIG. 10, it can be seen that the frame 110 is sized so that the scented disc 122 or other removable element that functions as a source of scent is positioned entirely in the interior 122 and preferably centrally disposed within the “T” shirt 134. That is, the “T” shirt 134 has a height 136 and a width 138 with the removable element or scented disc 122 having a center 123 centrally positioned about mid way of the height 136 and mid way of the width 138. It may also be noted that the removable element 122 is here shown to be circular or disc-like in shape. It may be made in any other geometric shape or form desired with the surface area selected to control the release rate of the scent and the volume of material selected to control the total amount of scent and, in turn, the useful life of the air freshener 10. The removable element 122 is also shown in FIG. 11 with a groove 124 sized to receive and register with the arms 116 and 118 as well as with the shoulders 112 and 114 to more securely hold the removable element 122 in place.

In connection with the configurations of FIGS. 10 and 11, it can be seen that the scent will dissipate and deplete over time. When the user wishes to replenish the air freshener and add new scent, the “T” shirt 134 may be lifted and the removable element or scented disc 122 removed and replaced with a new one. The “T” shirt 134 can then be repositioned over the frame 110 and over the removable element 122.

In FIG. 10, the suspension means is here shown to be a string or line 142 installed with knots to effect appropriate attachment to the frame 110. Alternately, the attachment means can include glue, adhesive strips, fasteners (e.g., nails, screws) or any suitable device to hold the air freshener in a desired location for a selected time, such as the useful life of the air freshener. Some attachment means may provide for portability or movability of the air freshener from one location to another.

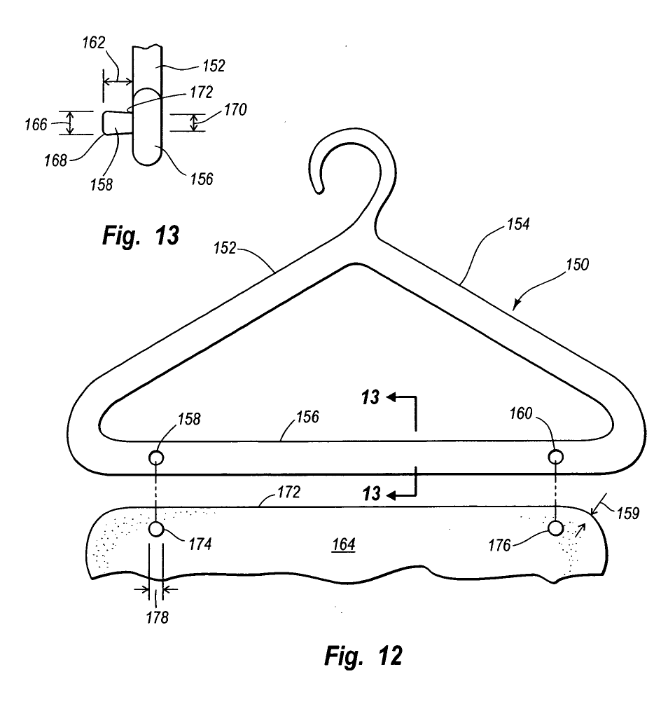

FIG. 12 shows a frame which is a hanger 150 much like hanger 52 of FIG. 6A. The hanger 150 has a left shoulder 152 and a right shoulder 154 sized to have an article like “T” shirt 12 suspended or hung thereon. The hanger has a pants bar 156 that is shown to be unitarily formed with the left shoulder 152 and the right shoulder 154. Of course, the pants bar may be formed or attached to or with the left shoulder 152 and right shoulder 154 in any suitable fashion so long as the pants bar 156 is adequately supported. Of course, the pants bar 156 may also be configured to be skirt hanger if desired.

The pants bar 156 has two pins 158 and 160 (attaching structure) formed therewith or assembled in any suitable way to extend generally normally away from the pants bar 156. The pins 158 and 160 may be unitarily formed with the pants bar 156 or they may be inserted and glued or frictionally held in suitable holes formed in the pants bar 156.

As better seen in FIG. 13, the pin 158 extends away a distance 162 selected to be greater than the thickness 159 and preferably at least twice the thickness of the source of scent 164. The pin 158 is here shown to be circular in cross section with a diameter 166 at its distal end 168 slightly larger than the diameter 170 at its proximal end 172.

The source of scent 164 may be made from scent impregnated plastic beads or from scent impregnated fiber board. It may be a solid or formed in a lattice comparable to the scented disc 122. The source of scent has an upper edge 172 and two holes 174 and 176 formed proximate the upper edge 172. The holes 174 and 176 have a diameter 178 selected so that when in register with their corresponding pins 158 and 160, the source of scent 164 must be pushed to frictionally pass onto the pins 158 and 160 and toward their proximal ends like proximal end 172 of pin 158. That is, pin 160 is comparable to pin 158. Because the source of scent 164 is somewhat deformable, it can deform so that the holes 174 and 174 can pass onto their respective pins 158 and 160. To remove, the source of scent is simply pulled off the pins 158 and 160.

It should be understood that the above illustrates what is presently regarded as preferred embodiments or configurations. Those of skill in the art will recognize that other embodiments can be constructed without deviating from the teachings herein while securing the advantages that evolve from the inventions hereinafter claimed.

What is claimed is:

1. An air freshener comprising:
an article proportionally miniaturized to be suspended decoratively within the cab of a vehicle, said article having an interior, said article having an aperture formed therein and said article being formed from a material having a porosity selected to regulate the passage of scent there through;
a frame sized to fit within said interior and configured to support said article, said frame having a source of scent configured to emanate scent therefrom and through said material to the exterior of the article; and
a suspension structure connected to said frame and extending through said opening for suspending said frame from a support structure in the cab of a vehicle.

2. The air freshener of claim 1 wherein said frame has shoulder structure from which said article is suspended.

3. The air freshener of claim 2 wherein said source of scent is said frame formed from scented plastic beads.

4. The air freshener of claim 1 wherein said source of scent is a removable element attached to said frame, said removable element having scent bearing material positioned for contact with the atmosphere.

5. The air freshener of claim 4 wherein said removable element is formed from scented plastic beads.

6. The air freshener of claim 4 wherein said frame has a retaining structure to removeably hold said removable element.

7. The air freshener of claim 3 wherein said retaining structure is sized to position said removable element centrally within the interior of said article.

8. The air freshener of claim 4 wherein said removable element is formed from paper impregnated with scent.

9. The air freshener of claim 1 wherein material is a non-woven material.

10. The air freshener of claim 1 wherein said material is a woven material.

11. The air freshener of claim 10 wherein said woven material includes cotton.

12. The air freshener of claim 11 wherein the woven material includes a synthetic material.

13. The air freshener of claim 12 wherein the synthetic material is any poly-resin.

14. The air freshener of claim 10 wherein said material is nylon.

15. The air freshener of claim 1 wherein said article is an article of clothing.

16. The air freshener of claim 15 wherein said article of clothing is a miniature “T” shirt.

17. An air freshener for use in a vehicle cab, said air freshener comprising:
an article of clothing miniaturized for suspension within the cab of a vehicle, said article of clothing having a front and a back with an interior therein between, said article of clothing having an aperture formed therein, and said article of clothing being formed from a material having a weave selected to regulate the passage of scent there through;
a frame sized to fit within said interior and to support said article of clothing, said frame being formed from a material impregnated with scent and shaped to support said article of clothing thereon; and
a suspension structure connected to said frame and extending through said opening for suspending said air freshener in said cab of a vehicle.

18. The air freshener of claim 17 wherein said suspension structure includes a loop sized to engage suspension structure in said cab.

19. The air freshener of claim 17 wherein suspension structure includes a line connected to said frame, said line having a length for securing said frame to a support.

20. The air freshener of claim 17 wherein said frame is formed from scented plastic beads.

21. The air freshener of claim 17 wherein said frame is formed of fiber board impregnated with scent.

22. An air freshener for use in a vehicle cab, said air freshener comprising:
a “T” shirt miniaturized for suspension decoratively within the cab of a vehicle, said said “T” shirt having an interior, said “T” shirt having an aperture formed therein, and said “T” shirt being formed from a woven material selected to regulate the passage of scent there through, said woven material being a blend that includes cotton;
a frame sized to fit within said interior and to support said “T” shirt, said frame being formed from scented material and shaped to support said article of clothing thereon; and
a suspension structure connected to said frame and extending through said aperture of said “T” shirt for suspending said air freshener in said cab of a vehicle.

23. The air freshener of claim 22 wherein said suspension structure includes a hook.

24. An air freshener comprising:
an article proportionally miniaturized for positioning in a closed space, said article having an interior, said article having an aperture formed therein and said article being formed from a material having a porosity selected to regulate the passage of scent there through;
a frame sized to fit within said interior and configured to support said article, said frame having a source of scent configured to emanate scent therefrom and through said material to exterior the article; and
a suspension structure connected to said frame and extending through said opening for suspending said frame from a support structure in said closed space.

25. The air freshener of claim 24 wherein said frame is formed from fiber board having scent impregnated therein.

26. The air freshener of claim 25 wherein said material is cloth formed from different fibers, one of which is cotton.

27. The air freshener of claim 24 wherein said frame has structure from which said article is supported, wherein said frame has attaching structure for connecting a source of scent to said frame, and wherein said source of scent is separate from said frame and attached thereto using said attaching structure.

28. The air freshener of claim 27 wherein said source of scent is a fiber board sheet impregnated with scent.

29. The air freshener of claim 27 wherein said source of scent is formed from scented plastic beads.

30. The air freshener of claim 27 wherein said frame is in the form of a miniature coat hanger with a pants bar, and wherein said attaching structure is formed with said pants bar.
